(19) United States
(12) Patent Application Publication
(10) Pub. No.: US 2025/0369874 A1
(43) Pub. Date: Dec. 4, 2025

(54) METHOD AND SYSTEM FOR ANALYSING ORE

(71) Applicants: ANGLO AMERICAN TECHNICAL & SUSTAINABILITY SERVICES LTD, London (GB); ANGLO CORPORATE SERVICES SOUTH AFRICA (PTY) LTD, Rosebank (ZA)

(72) Inventors: Colin Murray ANDREW, Rosebank (ZA); Daniel Clark FINFER, London (GB)

(21) Appl. No.: 18/875,030

(22) PCT Filed: Jun. 15, 2023
(86) PCT No.: PCT/IB2023/056209

§ 371 Date: Dec. 13, 2024

(30) Foreign Application Priority Data
Jun. 20, 2022 (GB) 2209023.7

(51) Int. Cl. G01N 21/31; E02F 9/26; E21C 39/00; G01N 21/71; G01N 21/85

(52) U.S. Cl. CPC G01N 21/31; E02F 9/26; E21C 39/00; G01N 21/718; G01N 2021/8592; G01N 2201/06113

(57) ABSTRACT

A method of analysing ore, wherein the method includes, during excavation, scanning/sensing ore, by using a sensor. The sensor is located at a position which is (i) spaced from a bucket/shovel of an excavator and (ii) operatively higher relative to the bucket/shovel. The sensor is also directed towards the bucket/shovel, so that the sensor can scan/sense operatively downwardly towards/into the bucket/shovel. The sensor may be secured to, or mounted on, an excavator at a position which is spaced from a bucket/shovel of the excavator.

### REFERENCES

AU 2020273300 relates to a mining shovel with compositional sensors which are installed inside a mining shovel bucket. These sensors may be photometric, radiometric or electromagnetic sensors.

EP 2 844 987 relates to a system and method of sorting mineral streams, for example laterite mineral ores, into appropriately classified valuable and waste streams for maximum recovery of value from the mineral stream. The method includes receiving response data indicating reflected, absorbed or backscattered energy from a mineral sample exposed to a sensor, where the mineral sample is irradiated with electromagnetic energy. The sensor may be an X-ray fluorescence sensor.

AU 2012277493 relates to a method of analyzing minerals received within a mining shovel bucket. The method includes collecting data associated with ore received in the bucket, where the bucket includes at least one active sensor which is installed in the bucket itself.

US2018/258608 describes an excavation machine which includes a front detection device and a rear detection device. The front detection device is mounted on a support member and detects a state of an object to be excavated by the excavator. The front detection device includes an imaging element such as a charge coupled device image sensor or a CMOS image sensor.

EP3748087 describes an excavator which uses a sensor to acquire the shape of the soil which is loaded in the bucket and estimate the amount of soil in the bucket, based on the image or point cloud data of the shape of the soil acquired by the sensor.

### BACKGROUND OF THE INVENTION

This invention relates to a method and system for analysing ore.

Bulk ore sorting (BOS) involves the use of sensing technology to reject waste ore prior to minerals processing for the purpose of reducing the energy intensity of the mining process and optimising the cost expended per tonne of metal produced. In conventional BOS, sensors are installed after the primary crusher to detect the grade of ore on the conveyor belt. A threshold can then be applied, making it possible to identify low grade ores and reject them to the waste pile or stockpile.

In other implementations, grade data is combined with other data, such as mineralogy or physical data, to determine the value of the ore, enabling Value-Based Ore Control (VBOC) which is more sophisticated than grade-based ore control. This mineralogical and physical data can be taken from the block model or, preferably, measured in real time in the post-crusher environment. In other implementations, sensor data from the belt can be used to stabilise the plant by determining the level of deleterious mineral present within a given sample. This plant stability method can be enhanced through the use of stockpiles. An example of stockpile-driven stability might be encountered in Copper mining where Arsenic is highly variable within the pit. A concentrate marketing philosophy might be developed to capitalise on stabilised Arsenic control, requiring the continual measurement and blending of feed to the plant for the purpose of targeting a specific do-not-exceed Arsenic level within the concentrate.

BOS to date has relied primarily on the use of on-belt sensors. These sensors come in a variety of types, and are defined typically by the geometric portion of the ore contributing to the output (volume or surface) and the type of data provided (chemical, mineralogical or physical). Volume sensors, which make it possible to represent the ore on a volume basis, offer the best representativity but are usually expensive nuclear devices (e.g. PFTNA (Pulsed fast and thermal neutron activation) or PGNAA (Prompt gamma neutron activation analysis)) or specialist systems available from a limited pool of suppliers (e.g. NMR (Nuclear Magnetic Resonance) systems). Surface sensors of several types (e.g. XRF, LIBS, or hyperspectral) are widely available from a large pool of suppliers, but can only measure what they can “see”, leading to lower representativity in typical installations in comparison to volume sensors. In terms of output type, chemical data can be used to determine grade, while mineralogical systems provide a picture of the actual type of rock present. Physical measurements can present many types of data (hardness, texture, density) and are important for contextualising both chemical and mineralogical data.

Up to now, the market has only presented one credible solution for sensing during excavation. This solution is based on XRF (X-ray fluorescence) technology and makes it possible to measure elemental composition of ore within the bucket during mining. This technology however has four primary technical drawbacks: (1) XRF sensing is limited to the measurement of surface properties, (2) XRF output is limited to chemical composition, (3) the existing XRF solution requires the sensors to be mounted in/on the mining buckets (owning to proximity requirements) which make them vulnerable to damage as rocks are being loaded into the bucket during operation, and (4) the proximity limits for this technology mean that there is likely no pathway to enabling the technology to future applications for scanning of the veneer during mining. An example of this technology is described in AU 2012277493 where sensor coils 815 are mounted inside the bucket 810. As a result the sensor coils 815 are vulnerable to damage as rocks are being loaded into the bucket.

The Inventors wish to address at least some of the problems mentioned above.

### SUMMARY OF THE INVENTION

In accordance with a first aspect of the invention there is provided a method of analysing ore, wherein the method includes:
during excavation, scanning/sensing ore, by using at least one sensor, wherein the at least one sensor is located at a position which is (i) spaced from a bucket/shovel of an excavator and (ii) operatively higher relative to the bucket/shovel and directed towards the bucket/shovel, so that the at least one sensor can scan/sense operatively downwardly towards/into the bucket/shovel.

It should be clear that the wording “during excavation” should be interpreted to include:
the loading of ore into the bucket/shovel,
the swing/movement of the bucket/shovel away from where the ore was loaded into the bucket, and
the dumping of the ore into a truck, onto the ground, into a crusher (fixed or mobile) or onto another piece of mining equipment.

Each one of these three actions therefore form part of excavation and the scanning/sensing can therefore take place during any of these actions.

The at least one sensor may be secured to, or mounted on, an excavator at a position which is spaced from a bucket/shovel of the excavator.

The at least one sensor may be a laser induced breakdown spectroscopy (LIBS) sensor.

The scanning/sensing step may include using two sensors to scan ore during excavation. The one sensor may be a laser induced breakdown spectroscopy (LIBS) sensor. More specifically, the scanning/sensing step may include using both a LIBS sensor and an active hyperspectral sensing (AHS) sensor.

Each sensor may be located at a position which is (i) spaced from the bucket/shovel of the excavator and (ii) operatively higher relative to the bucket/shovel and directed towards the bucket/shovel, so that the sensor can scan/sense operatively downwardly towards/into the bucket/shovel.

The scanning/sensing step may include scanning ore during excavation which is:
located in the bucket/shovel of the excavator, or in the process of entering into the bucket/shovel.

The at least one sensor may be mounted on a boom or arm of the excavator on which the bucket/shovel is mounted.

The method may include capturing images of the ore contained in the bucket/shovel or ore which is in the process of entering the bucket/shovel, from a position which is spaced from the bucket/shovel.

The method may include guiding a scanning/shooting pattern used/implemented by the at least one sensor, by utilising the captured images.

The imaging device may be mounted to a portion of the excavator which is spaced from the bucket/shovel, and wherein the images are captured using the imaging device.

The method may include utilising data/information obtained from the at least one sensor in order to determine ore value of ore which has been scanned/sensed.

The method may include utilising the determined ore value in order to determine:
where the scanned/sensed ore should be transported to; and/or
whether excavation operations should be focused at a particular area/location where the scanned/sensed ore was/is located.

The method may include communicating a decision as to (i) where the scanned/sensed ore should be transported to and/or (ii) whether excavation operations should be focused at the said particular area/location, to an operator of the excavator.

In accordance with a second aspect of the invention there is provided a system for analysing ore during excavation, wherein the system includes:
at least one sensor which is configured to scan/sense ore during excavation, wherein the at least one sensor is mounted on an excavator at a position which is (i) spaced from a bucket/shovel of the excavator and (ii) operatively higher relative to the bucket/shovel and directed towards the bucket/shovel, so that the at least one sensor can scan/sense operatively downwardly towards the bucket/shovel.

The at least one sensor may be a laser induced breakdown spectroscopy (LIBS) sensor.

The system may include two sensors which are configured to scan ore during excavation. The one sensor may be a laser induced breakdown spectroscopy (LIBS) sensor. The system may include a LIBS sensor and an active hyperspectral sensing (AHS) sensor which are both configured to scan ore during excavation.

Both sensors may be mounted on an excavator at positions which are (i) spaced from the bucket/shovel of the excavator and (ii) operatively higher relative to the bucket/shovel and directed towards the bucket/shovel, so that the sensors can scan/sense operatively downwardly towards/into the bucket/shovel.

The at least one sensor may be configured to scan ore during excavation which is:
located in the bucket/shovel of the excavator, or in the process of entering into the bucket/shovel.

The at least one sensor may be mounted on a boom or arm of the excavator on which the bucket/shovel is mounted.

The system may include an image capturing device which is mounted at a position which is spaced from the bucket/shovel and wherein the image capturing device is configured to capture images of the ore contained in the bucket/shovel or which is in the process of entering the bucket, when in use. The at least one sensor, or a controller/processor which is connected to the at least one sensor, may be configured to guide a scanning/shooting pattern used/implemented by the at least one sensor, by utilising the captured images.

The system may include a processing module which is configured to utilise data/information obtained from the at least one sensor in order to determine ore value of the ore which has been scanned/sensed by the at least one sensor.

A “module”, in the context of the specification, includes an identifiable portion of code, computational or executable instructions, or a computational object to achieve a particular function, operation, processing, or procedure. A module may be implemented in software, hardware or a combination of software and hardware. Furthermore, modules need not necessarily be consolidated into one device. The processing module may be configured to utilise the determined ore value in order to determine:
where the scanned/sensed ore should be transported to; and/or
whether excavation operations should be focused at a particular area/location where the scanned/sensed ore was/is located.

The at least one sensor may be configured to assess/determine both a minerology and a grade of the ore.

In accordance with a third aspect of the invention there is provided a method of analysing ore, wherein the method includes:
during excavation, scanning/sensing ore, by using at least one sensor, wherein the sensor is located at a position which is spaced from a bucket/shovel of an excavator.

The sensor may be secured to, or mounted on, an excavator at a position which is spaced from a bucket/shovel of the excavator.

The sensor may be a laser induced breakdown spectroscopy (LIBS) sensor.

The scanning/sensing step may include using two sensors to scan ore during excavation. The one sensor may be a laser induced breakdown spectroscopy (LIBS) sensor. The two sensors may be of the same type (e.g. both LIBS sensors) or they may be of different types (e.g. one LIBS sensor and one active hyperspectral sensing (AHS) sensor).

The scanning/sensing step may include scanning ore during excavation which is:
located in the bucket/shovel of the excavator,
or in the process of entering into the bucket/shovel.

The sensor may be positioned at an operatively higher position relative to the bucket/shovel, so that the sensor can scan/sense operatively downwardly towards/into the bucket/shovel.

The sensor may be mounted on a boom or arm of the excavator on which the bucket/shovel is mounted.

The scanning/sensing step may include scanning/sensing a veneer or muck during excavation, on which ore has been deposited or which includes ore.

The method may include capturing images of the ore contained in the bucket/shovel or ore which is in the process of entering the bucket/shovel, from a position which is spaced from the bucket/shovel.

The method may include guiding a scanning/shooting pattern used/implemented by the sensor, by utilising the captured images.

The method may include capturing images of the ore contained on a veneer or muck, during excavation.

An imaging device may be mounted to a portion of the excavator which is spaced from the bucket/shovel, and wherein the images are captured using the imaging device.

The method may include utilising data/information obtained from the sensor in order to determine ore value of ore which has been scanned/sensed.

The method may include utilising the determined ore value in order to determine:
where the scanned/sensed ore should be transported to; and/or
whether excavation operations should be focused at a particular area/location where the scanned/sensed ore was/is located.

The method may include communicating a decision as to (i) where the scanned/sensed ore should be transported to and/or (ii) whether excavation operations should be focused at the said particular area/location, to an operator of the excavator.

The communication step may include displaying information on a user interface which can be viewed by the operator.

The method may include assessing/determining a minerology and/or a grade of the ore, by using the at least one sensor.

In accordance with a fourth aspect of the invention there is provided a system for analysing ore during excavation, wherein the system includes:
at least one sensor which is configured to scan/sense ore during excavation, wherein the at least one sensor is mounted on an excavator at a position which is spaced from a bucket/shovel of the excavator.

In an alternative embodiment, the at least one sensor may be mounted on another type of excavation equipment.

The sensor may be a laser induced breakdown spectroscopy (LIBS) sensor.

The system may include two sensors which are configured to scan ore during excavation. The one sensor may be a laser induced breakdown spectroscopy (LIBS) sensor. More specifically, the system may include a LIBS sensor and an active hyperspectral sensing (AHS) sensor. Both sensors may be mounted on an excavator at positions which are spaced from the bucket/shovel of the excavator.

The sensor may be configured to scan ore during excavation which is:
located in the bucket/shovel of the excavator, or
in the process of entering into the bucket/shovel.

Alternatively, or in addition, the sensor may be configured to scan ore during excavation which is located on a muck, veneer or stockpile (i.e. at any excavation site).

The sensor may be positioned at an operatively higher position relative to the bucket/shovel, so that the sensor can scan/sense operatively downwardly towards the bucket/shovel.

The sensor may be mounted on a boom or arm of the excavator on which the bucket/shovel is mounted. The excavator may form part of the system.

The system may include an image capturing device which is mounted at a position which is spaced from the bucket/shovel. The image capturing device may be configured to capture images of the ore contained in the bucket/shovel or which is in the process of entering the bucket, when in use. The image data may be used to optimize a scanning operation of the system.

The sensor, or a controller/processor which is connected to the sensor, may be configured to guide a scanning/shooting pattern used/implemented by the sensor, by utilising the captured images.

The system may include a processing module which is configured to utilise data/information obtained from the sensors in order to determine ore value of the ore which has been scanned/sensed by the sensor.

The processing module may be configured to utilise the determined ore value in order to determine:
where the scanned/sensed ore should be transported to; and/or
whether excavation operations should be focused at a particular area/location where the scanned/sensed ore was/is located.

The system may include a communication module which is configured to communicate a decision as to (i) where the scanned/sensed ore should be transported to, and/or (ii) whether excavation operations should be focused at a particular area/location where the scanned/sensed ore was/is located. The communication module may be configured to display information on a user interface which can be viewed by the operator. The information may relate to the said decision.

The processing module may be configured to utilise the determined ore value in order to plan future excavation operations (e.g. where to transport the scanned ore to or whether excavation operations should be focused on an area where the ore was scanned).

The at least one sensor may be configured to assess both a minerology and a grade of the ore.

### BRIEF DESCRIPTION OF THE DRAWINGS

The invention will now be described, by way of example, with reference to the accompanying drawings. In the drawings:

FIG. 1A shows a three-dimensional view of a system for analysing ore, in accordance with the invention;

FIG. 1B shows an enlarged view of a mining bucket/shovel of the system, while ore contained in the bucket/shovel is being scanned by two sensors and images thereof are captured by a camera;

FIG. 2A shows a three-dimensional view of an alternative embodiment of the system in accordance with the invention;

FIG. 2B shows an enlarged view of a mining bucket/shovel of the system, while ore contained in the bucket/shovel is being scanned by one of the sensors (e.g. a LIBS sensor);

FIG. 2C shows an enlarged view of a mining bucket/shovel of the system, while ore contained in the bucket/shovel is being scanned by a first sensor (e.g. a LIBS sensor), followed by a second sensor (e.g. an AHS sensor); and

FIG. 3 shows a schematic layout of the system in accordance with the invention.

### DESCRIPTION OF PREFERRED EMBODIMENTS

The present invention aims to address ore sensing during excavation, by using standoff optical technology. The optical technology can be installed on an excavator 12 at a location(s) which is/are mechanically decoupled from a bucket/shovel 14 of the excavator 12 (i.e. not installed in or on the bucket itself). For the sensing/scanning, two sensors/sensor arrays 16, 18 are used (although only one sensor could also be used) in order to sense both grade and mineralogy of ore 100. In one specific application, the sensors can be used to sense/scan ore contained inside the bucket 14 of the excavator 12, or ore 100 which is in the process of entering the bucket 14, using a whisk-broom approach. However, it should be noted that it may also be possible, in future, to use a push-broom approach or possibly even snapshot implementations.

In another application, other areas of an excavation site can also be scanned by the sensors 16, 18 during excavation (i.e. not just ore contained inside the bucket 14). These areas may include a muck, a veneer, a stockpile, or any other area still to be excavated.

The sensor(s) 16, 18 could be configured such that it can be moved and placed at different positions (e.g. to scan different parts of a veneer/stockpile 102). Alternatively, the sensor(s) 16, 18 could be installed on a vehicle/machine which can then move the sensor(s) 16, 18 to different areas at an excavation site in order to scan different portions of the ore 104. Alternatively, the sensor(s) 16, 18 could be installed on a vehicle/machine which can then move the sensor(s) 16, 18 to different areas at the excavation site in order to scan different portions of the ore.

It should also be noted that the sensor(s) 16, 18 could be mounted on a raised stanchion or observation pole or structure, and look down into an operating pit. More specifically, the sensor(s) 16, 18 may be mounted on a structure (i.e. other than an excavator) and directed towards a muck, a veneer, a stockpile, or any other area still to be excavated.

The present invention allows ore properties to be determined, despite being physically remote from the region of rock movement which accompanies mining excavation (e.g. the sensors 16, 18 are mounted to a boom 20 of an excavator 12 and can be directed downwardly towards the bucket 14 of the excavator 12 or be directed towards a muck or veneer).

Two types of sensors can be used as primary sources of data, namely a laser induced breakdown spectroscopy (LIBS) sensor 16 and a hyperspectral sensing sensor. More specifically, the hyperspectral sensing sensor is an active hyperspectral sensing (AHS) sensor 18.

With LIBS sensors, a laser pulse is used to ablate an amount of material on the surface of a sample and breaks it down into plasma consisting of atoms, ions and free electrons. The chemical composition of a material can be characterised by measuring the radiative emission of the cooling plasma with a spectrometer. LIBS sensors therefore enable remote sensing of chemical compositions of a sample (e.g. ore contained in a bucket), whereby the sample and sensor can be relatively far apart (e.g. 5-30 m). LIBS sensing can typically be applied to various forms of matter, including liquids and slurries. A LIBS sensor is capable of returning results in a split second and can also be used on moving samples. The operation of LIBS sensors is well known and will therefore not be described in more detail. However, reference is specifically made to the following document (see specifically pages 57-59 which describes the operation of LIBS sensors) which is incorporated herein by reference:
Dalm, Marinus, Sensor-based sorting opportunities for hydrothermal ore deposits: Raw material beneficiation in mining, TUDelft, Delft University of Technology, 2018 (https://repository.tudelft.nl/islandora/object/uuid: 70a1e180-ef0c-4226-9af3-7e9dc3938c7f?collection=research)

AHS sensors use a controlled hyperspectral beam to generate a spotlight which can be carefully monitored to identify certain types of minerals (based on reflected or scattered light which is detected). The operation of hyperspectral sensing, and more specifically, AHS sensing is a known technology which has been described by others in the field. Reference is in this regard made to the following publication which is incorporated herein by reference:
Kääriäinen, Teemu, et. al., Active Hyperspectral Sensor Based on MEMS Fabry-Péron Interferometer, 2019 (Published 12 May 2019), Sensors, MDPI https://www.researchgate.net/publication/333046518_Active_Hyper spectral_Sensor_Based_on_MEMS_Fabry-Perot_Interferometer

Both LIBS and AHS employ a whisk-broom approach (although it is likely that AHS may, in future, use a push-broom approach), meaning that each sensor 16, 18 produces a beam (see reference numerals 22 and 24 which illustrates these beams schematically) which must be scanned over the surface of interest (e.g. the surface of the ore 100 exposed inside the bucket 12 or of ore which is in the process of entering the bucket 12; or the muck/veneer/other area still to be excavated) to produce the desired output. The desired output can then be used (1) for fleet management and (2) feed-forward control, both of which are explained in more detail further below.

In one embodiment, a LIBS sensor(s) could be used to assess grade, while an AHS sensor could be used to determine minerology. In an alternative embodiment, the LIBS sensor could be used to determine minerology and an AHS sensor could be used to determine grade. In a further alternative embodiment, a LIBS sensor(s) could be used to assess both grade and minerology.

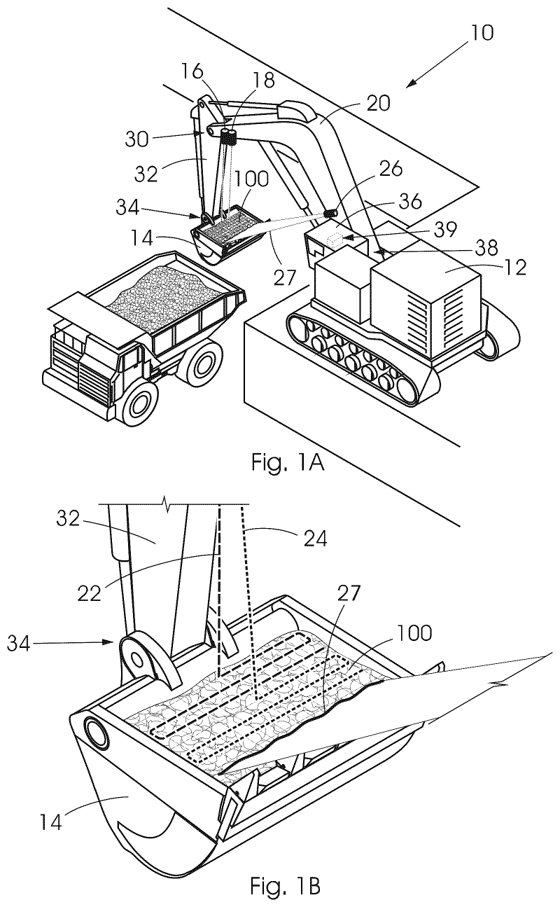

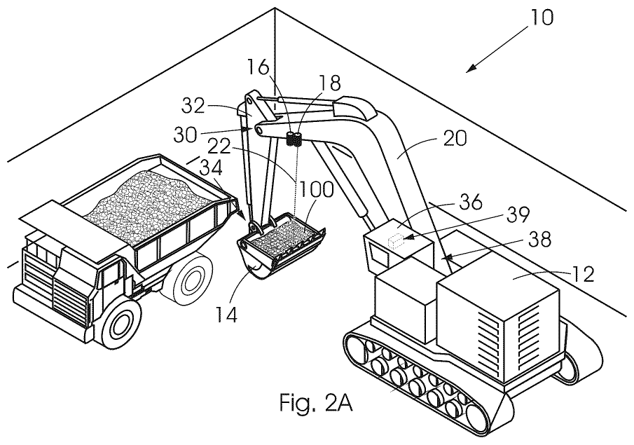

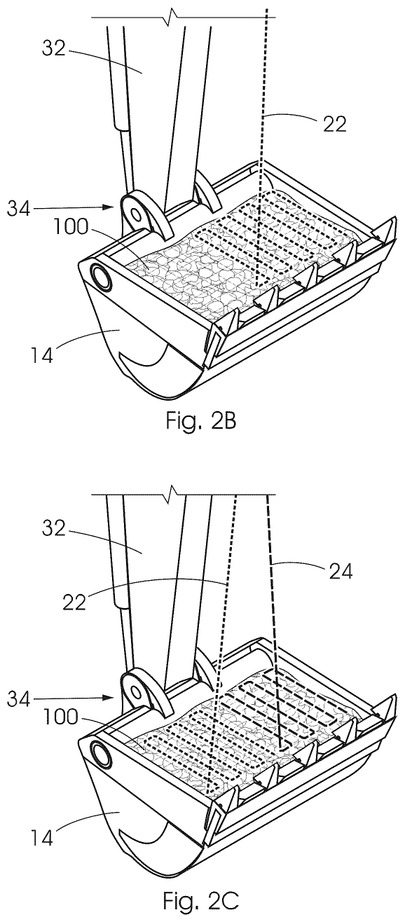

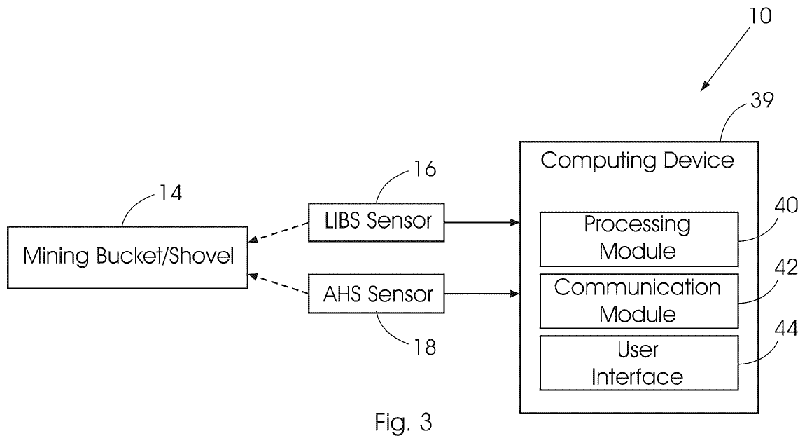

The present invention may also implement a photographic method(s) to enable mine operators to gather a sophisticated picture of ore 100 as it is being excavated. In this regard, reference is made to FIGS. 1A and 1B which illustrates how an imaging device 26, such as a camera, is used to capture images of the ore 100 contained inside the bucket 14 (see reference numeral 27 which illustrates the capturing schematically). The imaging device 26 may however also be used to capture images of the ore 100 as it is in the process of entering the bucket 14. FIGS. 2A-2C however show an alternative embodiment of the invention where an imaging device is not included. It should be noted that the camera 26 may also be used to control the whisk-broom or push-broom sampling pattern of the sensors 16, 18. More specifically, the sensor 16, 18, or a controller/processor which is connected to the sensors 16, 18, may be configured to guide a scanning/shooting pattern used/implemented by the sensors 16, 18, by utilising the captured images.

FIGS. 1A and 1B show a first example of an installation of a system 10 in accordance with the invention for in-pit sensing. As shown, the system 10 includes the two optical sensors 16, 18 (i.e. the LIBS and AHS sensors 16, 18) which are mounted on a boom 20 of the excavator 12, so that there is no/little risk of interference by falling rock during excavations. More specifically, the two sensors 16, 18 are mounted close to a first end 30 of the boom 20 where an arm/dipper/stick 32 of the excavator 12 is pivotally connected to the boom 20. This allows the sensors 16,18 to sense downwardly into the bucket 14 of the excavator 12, whereby the bucket 14 is mounted to a lower free end 34 of the arm/dipper/stick 32, in order to measure ore properties of the ore 100 inside the bucket 14.

By using sensors 14, 16 with the ability to measure ore properties at a standoff (i.e. at a distance), such as LIBS and AHS sensors, it is possible to install the sensors 14, 16 in a zone free from rockfall (e.g. there is no need to install the sensors 14, 16 inside the bucket 14). Further, by using standoff sensors 14, 16, it is possible to scan the muck, veneer, mine face or even truck. Furthermore, by using both a UV-based LIBS sensor 16 and an IR-based AHS sensor 18, it becomes possible to measure not only grade but also mineralogy, which leads to the opportunity to measure ore value, which is a more sophisticated control quantity than grade alone. In this regard, it should be noted that ore value is a function not only of grade, but also of mineralogy and physical characteristics. For example, two rocks may be of the same grade, but one of them may have a higher hardness and will therefore require more energy intensive processing. As a result, the “harder” rock will be lower in value, when compared to the other rock.

This sensing capability can be complemented by camera imaging (see FIGS. 1A and 1B) to enable identification of texture and lithology. More specifically, the system 10 may include a camera 26 which is mounted on the boom 20, near a cab 36 of the excavator 12 (i.e. near a second end 38 of the boom 20 (opposite the first end 30)), and which is directed to capture images of ore 100 contained in the bucket 12.

### Example

As will be clear from the explanation above, the system 10 allows for sensing during excavation. When an excavator 12 is used to pick up ore 100, the sensors 16, 18 are used to scan the ore 100 inside the bucket 14 (see FIGS. 1B and 2B), in order to measure the ore's grade and minerology. A top of the bucket 14 is therefore scanned, and the grade of the bucket volume is extrapolated statistically. The ore value can then be determined subsequently, by making use of these measurements and calculations.

For example, the system 10 may include a computing device/system 39 which includes a processing module 40 and a communication module 42. The data/information obtained by the sensors 16, 18 is sent to the communication module 42 via a communication network/link (e.g. via wireless communication, such as Bluetooth). The processing module 40 then utilises the data/information to calculate the ore value. The computing device 39 may be located on the excavator 12 or could be located at a remote location, e.g. a monitoring station. Based on the measured grade, minerology and/or ore value, a decision can be made as to whereto the ore 100 contained in the bucket 14 should be transported. For example, lower grade ore could be transported to a waste pile or stockpile, while higher grade ore could be transported to the crusher or leach pad for further processing.

The indication of the ore value can typically be communicated to an operator via a user interface 44. In one example, the user interface 44 may form part of the computing device 39 and may be installed inside the cab 36. In a slight alternative example, the computing device 39 could send the indication of the ore value to a separate user interface 44 (e.g. via wireless communication) installed inside the cab 36 (e.g. the user interface 44 may include a display screen on which information is displayed). The user interface of would then indicate to the operator (e.g. on a display screen) what to do with the ore inside the bucket, depending on the ore value/grade. For example, the scanned ore should be transported to a particular location for further processing), depending on the ore value.

It should however be noted that the sensors 16, 18 could, as an alternative, scan the ore as it is in the process of entering the bucket 14 (e.g. rocks are scanned as they enter the bucket 14). The bucket could therefore be shaped to enhance the ability for the sensors 16, 18 to maximise rock visibility as measured in terms of overall surface area for the bucket contents. This enhancement might make use of unique bucket shapes to increase preferably the amount of material that can be scanned. Further, loading technique could be used to enhance the “roll” of rocks into the bucket 14, leading to higher overall surface area viewed.

In another variation, the range of the sensors 16, 18 could be extended further so that the veneer and/or muck can be scanned during the course of bucket loading operations. Based on the scanning, an operator can then, based on the ore value, determine if excavation operations should be focused at the particular scanned area (e.g. based on high ore value) or whether the excavation operations should rather focus on other areas (e.g. based on low ore value).

It should be noted that the system of the present invention can be used to deliver fleet management instructions, provide a priori information for conventional belt-based bulk ore sorters, calibrate the mine control ore body model, and anticipate changes in the plant via feed-forward control.

It should be noted that with bulk sorters, not all BOS (bulk ore sorting) sensing is deterministic. A given BOS sensor might measure one aspect of value (such as clay content) and rely on the input of LIBS data for grade. Alternatively, clay and geographic data obtained during excavation might feed-forward to inform a BOS system which proxy to use if direct grade measurement is not possible. Proxy guidance could be critical in PGM's (Platinum Group Metals), where grade is measured in PPM (parts per million).

Plant stabilization might be done using talc data from the AHS sensor. Talc tends to cause overflow in the flotation plant. If talc can be detected ahead of processing, mitigation action can be taken by changing the reagent concentration in the plant.

1-23. (canceled)

24. A method of analysing ore, wherein the method includes:
during excavation, scanning/sensing ore, by using at least one sensor, wherein the at least one sensor is located at a position which is (i) spaced from a bucket/shovel of an excavator and (ii) operatively higher relative to the bucket/shovel and directed towards the bucket/shovel, so that the at least one sensor can scan/sense operatively downwardly towards/into the bucket/shovel.

25. The method of claim 24, wherein the at least one sensor is secured to, or mounted on, an excavator at a position which is spaced from a bucket/shovel of the excavator.

26. The method of claim 25, wherein the at least one sensor is a laser induced breakdown spectroscopy (LIBS) sensor.

27. The method of claim 25, wherein the scanning/sensing step includes using both a laser induced breakdown spectroscopy (LIBS) sensor and an active hyperspectral sensing (AHS) sensor, and wherein each sensor is located at a position which is (i) spaced from the bucket/shovel of the excavator and (ii) operatively higher relative to the bucket/shovel and directed towards the bucket/shovel, so that the sensor can scan/sense operatively downwardly towards/into the bucket/shovel.

28. The method of claim 26, wherein the scanning/sensing step includes scanning ore during excavation which is:
located in the bucket/shovel of the excavator, or
in the process of entering into the bucket/shovel.

29. The method of claim 26, wherein the at least one sensor is mounted on a boom or arm of the excavator on which the bucket/shovel is mounted.

30. The method of claim 28, which includes capturing images of the ore contained in the bucket/shovel or ore which is in the process of entering the bucket/shovel, from a position which is spaced from the bucket/shovel.

31. The method of claim 30, which includes guiding a scanning/shooting pattern used/implemented by the at least one sensor, by utilising the captured images.

32. The method of claim 31, wherein an imaging device is mounted to a portion of the excavator which is spaced from the bucket/shovel, and wherein the images are captured using the imaging device.

33. The method of claim 26, which includes utilising data/information obtained from the at least one sensor in order to determine ore value of ore which has been scanned/sensed.

34. The method of claim 33, which includes utilising the determined ore value in order to determine:
where the scanned/sensed ore should be transported to; and/or
whether excavation operations should be focused at a particular area/location
where the scanned/sensed ore was/is located.

35. The method of claim 34, which includes communicating a decision as to (i) where the scanned/sensed ore should be transported to and/or (ii) whether excavation operations should be focused at the said particular area/location, to an operator of the excavator.

36. A system for analysing ore during excavation, wherein the system includes:
at least one sensor which is configured to scan/sense ore during excavation, wherein the at least one sensor is mounted on an excavator at a position which is (i) spaced from a bucket/shovel of the excavator and (ii) operatively higher relative to the bucket/shovel and directed towards the bucket/shovel, so that the at least one sensor can scan/sense operatively downwardly towards the bucket/shovel.

37. The system of claim 36, wherein the at least one sensor is a laser induced breakdown spectroscopy (LIBS) sensor.

38. The system of claim 36, wherein the system includes a laser induced breakdown spectroscopy (LIBS) sensor and an active hyperspectral sensing (AHS) sensor which are both configured to scan ore during excavation, and wherein both sensors are mounted on an excavator at positions which are (i) spaced from the bucket/shovel of the excavator and (ii) operatively higher relative to the bucket/shovel and directed towards the bucket/shovel, so that the sensors can scan/sense operatively downwardly towards/into the bucket/shovel.

39. The system of claim 36, wherein the at least one sensor is configured to scan ore during excavation which is:
located in the bucket/shovel of the excavator, or
in the process of entering into the bucket/shovel.

40. The system of claim 36, wherein the at least one sensor is mounted on a boom or arm of the excavator on which the bucket/shovel is mounted.

41. The system of claim 36, which includes an image capturing device which is mounted at a position which is spaced from the bucket/shovel and wherein the image capturing device is configured to capture images of the ore contained in the bucket/shovel or which is in the process of entering the bucket, when in use, and wherein the at least one sensor, or a controller/processor which is connected to the at least one sensor, is configured to guide a scanning/shooting pattern used/implemented by the at least one sensor, by utilising the captured images.

42. A method of analysing ore, wherein the method includes:
scanning different areas at an excavation site using at least one sensor, whereby a vehicle is used in order to move the at least one sensor to the different areas at the excavation site,
wherein the at least one sensor is a laser induced breakdown spectroscopy (LIBS) sensor.

43. The method of claim 42, wherein the method includes:
providing two sensors on the vehicle, wherein the one sensor is a laser induced breakdown spectroscopy (LIBS) sensor and the other sensor is an active hyperspectral sensing (AHS) sensor, and
scanning the different areas at the excavation site using the two sensors, whereby the vehicle is used in order to move the two sensors to the different areas at the excavation site.
